(19) United States
(12) Patent Application Publication
(10) Pub. No.: US 2023/0317211 A1
(43) Pub. Date: Oct. 5, 2023

(54) METHOD AND SYSTEM FOR ENCRYPTING GENETIC DATA OF A SUBJECT

(71) Applicants: ASSISTANCE PUBLIQUE HOPITAUX DE MARSEILLE, MARSEILLE (FR); UNIVERSITÉ D'AIX-MARSEILLE, MARSEILLE (FR)

(72) Inventors: Frédéric FINA, GREASQUE (FR); Alain BIANCOTTO, MARSEILLE (FR); Eric PELLEGRINO, SAINT SAVOURNIN (FR); Maéva DELAVEAU, AUBAGNE (FR); Nicolas MACAGNO, MARSEILLE (FR); Dominique FIGARELLA-BRANGER, CASSIS (FR)

(21) Appl. No.: 18/019,277

(22) PCT Filed: Aug. 2, 2021
(86) PCT No.: PCT/EP2021/071531

§ 371 Date: Feb. 2, 2023

(30) Foreign Application Priority Data
Aug. 3, 2020 (EP) 20305891.2

(51) Int. Cl. G16B 50/40; H04L 9/08

(52) U.S. Cl. CPC G16B 50/40; H04L 9/0866

(57) ABSTRACT

A computer implemented method and a system of encryption of genomic data of a biological sample are provided, that improve the security of genetic information obtained from a sample, while guaranteeing traceability and identity-vigilance throughout the analysis chain. The computer implemented method and system disclosed herein allows a high level of identity-vigilance, improved labelling and traceability and provide a high level of confidentiality of genomics data.

### FIELD

The present disclosure relates to a computer implemented method and a system of encryption of genomic data of a biological sample and DNA labelling of the same.

### BACKGROUND

The evolution of DNA sequencing technologies over the past decades has allowed sequencing a subject's whole genome at a relatively low cost. Hundreds of thousands of subjects have hence contributed samples to sequencing laboratories, either for personal purpose (for example genealogical DNA tests), for medical reasons or also for translational research.

Personalized medicine is the future of health care, as whole-genome sequencing provides the ability to personalize treatment at the individual level and stage of his or her disease.

Because pharmacology and drug development are based on population studies, current treatments are standardized to whole population statistics. However, a subject's response to disease and drug therapy is related to his or her genetic and epigenetic predisposition.

Genome sequencing has accelerated prognostic counselling in monogenic diseases, where rapid and differential diagnosis in neonatal care is important. However, the often blurred distinction between medical and research use can complicate the way in which confidentiality between these two areas is handled, as they often require different levels of consent and involve different national policies. Moreover, these policies are very different between Europe, where the attitude is towards the protection of the subject's data, and Anglo-Saxon countries, where the attitude is towards the liberalisation and distribution of data.

Indeed, corporate privacy policies are often not under national jurisdiction, particularly in Anglo-Saxon countries, which exposes consumers to information risks, both with regard to their genetic data and to their disclosed consumer profile, including family history, health status, race, ethnicity, social networks, etc. For example, certain companies are selling collected genomics data to industrialists or are sharing them in public databases, biobanks and repositories (e.g. UK biobank and the 1000 Genomes Project) to assist researchers and clinicians to advance biomedical research, to better understand the structures and functionalities of biological data-DNA, RNA and proteins.

Given that the nature of consumer transactions allows these electronic models to bypass traditional forms of consent in research and health care, policy on the protection of genetic personal information is even more complicated. The same applies when considering international research collaborations or biological resource centres (international biobanks), databases that store biological samples and genetic information.

In addition, research and health care are not the only areas that require formal expertise; other areas of concern include the privacy of genetic information of those involved in the criminal justice system and those involved in private, consumer-oriented genomic sequencing.

Pharmaceutical industries with insurance companies, employers or potentially eugenic totalitarian states are the main sources of concern. Consumers may not fully understand the implications of digitizing and storing their genetic sequence. It is therefore important to stress that in the event of a data breach, an subject's personal genome cannot be replaced. The priority then is to determine which methods are robust and how policies should ensure continued genetic privacy.

There are thus serious concerns about the security and privacy of genomic data in storage, sharing, in transit and during computation. One can indeed imagine laws allowing States or private companies to have access to the genomics data stored in these databanks.

In order to address these concerns, different cryptographic strategies have been proposed. For example, it has been proposed to divide the reading mapping in two tasks: the matching of the sequencing data which can be performed on a public cloud, while the alignment of these readings is performed on a private cloud. However, since the alignment processes tend to be very large and labour-intensive, most sequencing systems still functionally require third-part computing operations such as clouds, which pose security concerns.

Other studies have proposed a technique that uses homomorphic encryption and a secure full comparison, and suggests storing and processing sensitive data in encrypted form. To ensure confidentiality, the Storage and Processing Unit (SPU) stores all the single nucleotide polymorphisms (SNPs) observed in the patient with redundant content from a set of potential SNPs. Another solution has developed three protocols to secure the calculation of mounting distances using Yao's Garbled circuit intersections and a strip upgrade algorithm. However, the major disadvantage of this solution is its inability to perform large-scale calculations while maintaining accuracy.

Also, in NGS analyses, sequences called Tag or MID are added at the time of library preparation during the analytical phase. These sequences are carried in 3′ by the PCR primers, during demultiplexing the obtained sequences are aligned with the reference sequences of the target genome, the 3′ part allows to identify the samples for each sequence aligned in the same sequencing assay (run). These tags or MIDs are reused in each new run and index the new samples in the following analysis series (new run). These tags or MIDs are not unique and no numerical data is encoded in the base sequence.

To date, there is no solution combining the reading by sequencing of biological information and digital data encoded using the 4 ATGC bases and encrypted on a custom-produced nucleic acid support, forming a unique invariant, and carrying information of the following types: indexing data, clinical data, biological data, personal data, images, etc.

Moreover, it is not currently possible to give patients autonomy (choice) as to the use of their genomic data by a third-part. Also, it is difficult to stratify patient consent according to the level of genomic information that is strictly necessary for analysis.

### BRIEF DESCRIPTION OF THE DRAWINGS

FIG. 1 represent a chart flow of the method disclosed herein.

FIG. 2 represents an illustration of the encryption method by blocks of a raw data “FASTQ” file.

### LIST OF ABBREVIATIONS

BAM=Binary Alignment Map
DNA=Deoxyribonucleic Acid
HER=Electronic Health Record
HLA=Human Leukocyte Antigen
QC=Quality Control
MDD=Metadata Document
MID=Multiplex Identifier
NGS=Next-Generation Sequencing
PCR=Polymerase Chain Reaction
RNA=Ribonucleic Acid
SNP=Single-Nucleotide Polymorphism
SPU=Storage and Processing Unit

### SUMMARY

Embodiments described therein provide a computer implemented method for encrypting genetic data of a subject, comprising the following steps:
Step a) synthetizing, by a DNA synthesiser, an exogenous DNA sequence (DNA tag) comprising encoded metadata relating to said subject, said metadata comprising at least an encryption key, said encryption key being unique and associated to said subject;
Step b) collecting a biological sample of said subject in a sampling material, said sampling material comprising said exogenous DNA sequence;
Step c) sequencing, by a DNA sequencer, the DNA of said subject obtained from said biological sample and sequencing, by a DNA sequencer, said exogenous DNA sequence comprising encoded metadata,
Step d) creating by at least one processing unit a text-based file corresponding to the sequenced genome of the subject, said genome comprising at least one sequence of interest,
Step e) creating by said least one processing unit a text-based file corresponding to the sequenced exogenous DNA sequence comprising encoded metadata comprising at least an encryption key;
Step f) extracting by means of said least one processing unit the encryption key from said text-based file corresponding to the sequenced exogenous DNA sequence;
Step g) encrypting by said least one processing unit said text-based file corresponding to the sequenced genome of the subject with said encryption key from step f) associated to said subject, apart from the at least one sequence of interest.
   The method may further include one and/or other of the following features:
In step a), said metadata comprise at least a second encryption key
the at least one sequence of interest is encrypted in step g) by means of said second encryption key;
the text-based file of step d) is fragmented in blocks of fixed-length base pairs;
encoding a personal database index identifier associated to said subject within the exogenous DNA sequence;
encoding information to identify the at least one sequence of interest within the exogenous DNA sequence.
encoding the health record of the subject within the exogenous DNA sequence;
encoding metadata in the exogenous DNA sequence in the form of a binary code based on the combination of the 4 nucleotide bases A, T, G and C;
encrypting the metadata encoded within the exogenous DNA sequence with a third encryption key.
   A system for encrypting genetic data of a subject is also provided, comprising:
(a) a DNA synthesizer configured to synthetize an exogenous DNA sequence comprising encoded metadata relating to said subject, said metadata comprising at least an encryption key, said encryption key being unique and associated to said subject;
(b) a DNA sequencer configured to sequence said exogenous DNA sequence comprising encoded metadata relating to said subject and configured to sequence the DNA of said subject obtained from a biological sample;
(c) at least one processing unit configured to perform the following steps:
   creating a text-based file corresponding to the sequenced genome of the subject, said genome comprising at least one sequence of interest;
   creating a text-based file corresponding to the sequenced exogenous DNA sequence, the sequence of exogenous DNA sequence comprising encoded metadata comprising at least an encryption key;
   extracting the encryption key from the text-based file corresponding to the sequenced exogenous DNA sequence;
   encrypting the text-based file corresponding to the sequenced genome of the subject with said encryption key.
      The system may further include one and/or other of the following features:
at least one additional processing unit configured to perform the following steps:
   convert the metadata comprising at least an encryption key into a binary code based on the combination of the 4 nucleotide bases A, T, G and C so as to obtain a nucleic acid sequence corresponding to said metadata;
   transmitting the obtained nucleic acid sequence to the DNA sequencer so as to obtain the exogenous DNA sequence comprising encoded metadata comprising at least said encryption key.
at least one processing unit configured to fragment the text-based file corresponding to the sequenced genome of the subject in blocks of fixed-length base pairs.

Thanks to these dispositions, the method and system improve the security of genetic information obtained from a sample, while guaranteeing traceability and identity-vigilance throughout the analysis chain. The “identity-vigilance” aims to ensure that all subjects are correctly identified throughout the analysis process (for e.g. when the subject is a patient, throughout their care in the hospital and in the exchange of medical and administrative data). The objective is to make subject identification and documentation reliable throughout the entire course of care so that the right care, to the right subject, at the right time can always be provided.

The method and system disclosed herein allows a high level of identity-vigilance because since the label sequence includes the subject's information, and since it is in the same tube as the sample to be analysed, it is possible to determine a subject's identity in a secure manner and thus avoid, for example, misdiagnosis when the subject is a patient. It can also be compared with data stored conventionally in digital format, thus ensuring quality control of the data.

Moreover, labelling and traceability are improved. Indeed, based on the same principle of having the label sequence in the same tube as the sample, it is possible to have a labelling of the sample years later. Thus, the problem of data loss linked to a sample (label removal or fading) is solved in this way.

Furthermore, through this DNA tag coding for metadata comprising at least a cryptographic key, only the holders of the key (client) or of the original sample (laboratory in charge of sequencing the genome) are able to decipher the subject's genome stored in the laboratory databank.

### DETAILED DESCRIPTION

In the Figures, the same references denote identical or similar elements.

The method and system disclosed therein provides performance gain and new use for “identity-vigilance” as well as a new use for “encoding” digital data such as, for e.g. health data. Improved security and privacy of biologic data is also provided by the present method. Indeed, identity-vigilance begins at the time of sampling, in combination with the other quality controls (QC) usually used throughout the analytical chain.

Also, encoding makes it possible to combine private and genomic data on a physical medium. It makes it possible to keep in addition to digital data, a physical medium of these data re-analysable very robust in time, beyond all existing digital media (>2000 years).

In addition, encryption makes it possible to preserve one's personal autonomy, to give back to every human the property of his own person (J. Locke) and his freedom of individual choice. It also allows protecting any genomic data from biologic material, whatever these genomic data are from a human, an animal, bacteria, yeast or a vegetal.

Finally, indexing of the different levels of confidentiality of the genome, for the deciphering, reduces the size of the genome and thus the analysis time.

To do so, data are encoded in a synthetic exogenous DNA sequence, using the 4 nucleotide bases, like the binary coding used in computing, e.g. ‘00’=‘A’; ‘01’=‘T’, ‘01’=‘C’, ‘10’=‘G’. The exogenous DNA sequence is for e.g. synthetized by means of a DNA synthesizer. The data is stored in this unique DNA molecule (DNA tag or label) which is custom-made.

The DNA tag refers to the biological sample and/or its subject. The subject can be a human, an animal, bacteria, yeast or even a plant. The DNA tag is the physical carrier of digital information relating to the subject. The DNA label permanently accompanies the biological sample in a physical manner and the data derived from it in a digital manner.

Any sort of data relating to the subject can be encoded within the DNA tag. Said data can be for example any information relating to the identity of the subject (e.g. name, barcode, database identification number, etc.); to the sample collection conditions (e.g. date and place); to the nature of the sample (e.g. blood sample taken from a patient with specified condition) or even, in the case of a patient, to the patient's medical record.

The DNA tag further encodes for at least a cryptographic key which will be used to encrypt the genomic data obtained from the sample; or for metadata (MDD) indicating which parts of the genome are to be crypted. The cryptographic key encoded within the DNA tag is a public key and is associated to a private key. Said private key is unique, associated to the subject, confidential and only the client who is ordering the analysis has it in his possession.

In a general manner, all information relating to the subject can be encoded in the DNA tag in order to ensure privacy of personal/sensitive informations. Therefore, only a person in possession of the sample and being able to sequence DNA can have access to these informations, contrary to usual informations written on a label.

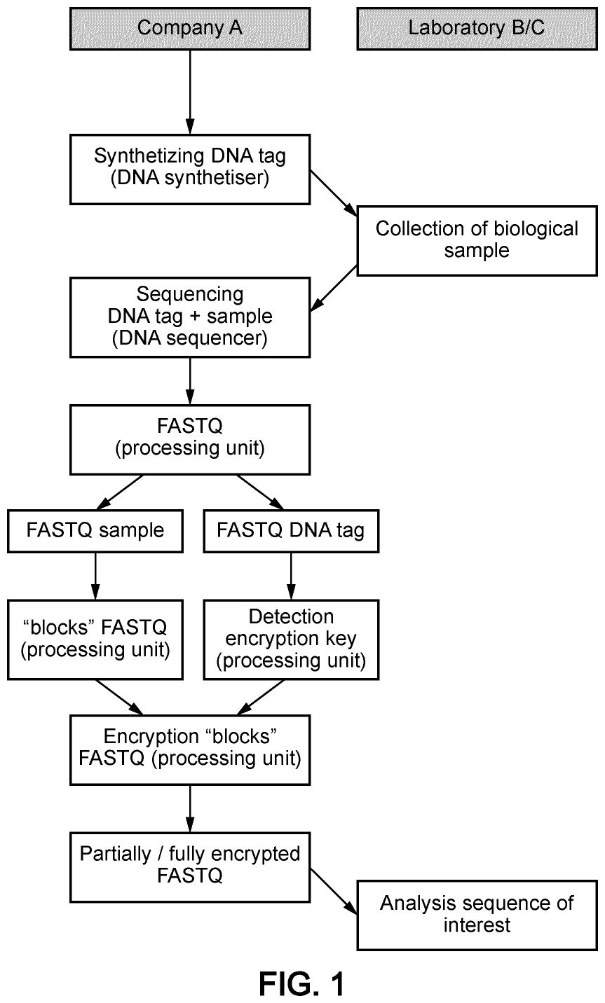

In the present method, the DNA tag is added to the sample at the time of its collection. It is then read by a sequencer, along with the biological data from the genome of the subject, present in the sample. The chart flow of the present method is illustrated in FIG. 1.

The data present on the DNA tag thus serves different purposes: identity monitoring, annotations but also securing the sample by serving as a physical support for an encryption key.

The label is the physical support to the cryptographic public key, which indexes and deciphers different levels of “risks”. It is the physical key encrypting the genome of the subject, itself encrypted with the same security standards as current computer systems. The exogenous sequence can be encrypted by means of a third encryption key, chosen by the client ordering the analysis (e.g. a patient, agronomy industrial, laboratory, etc). Therefore, to obtain the translation of the information related to the subject, it is necessary to have the key which is held by the client.

The different level of risks are defined following the different levels of risk are defined according to the sequences relevant or not for the analysis. For example, it can be decided to encrypt only the sequences irrelevant for such analysis. Therefore, only the relevant sequences for the analysis are “readable” by a third-part while the rest of the genome is protected. It may also be decided to encode the relevant parts by means of a second key, which will be communicated to third-parts for deciphering (eg.g. the laboratory in charge of the analysis of the sequence of interest).

Therefore, only a person in possession of the original sample containing the DNA tag and/or the private key are able to decipher the entire subject's genome. The label is the “physical” lock on the subject's data, protecting it from hacking, theft or misuse of these genomic and private data. To obtain the translation of the information related to the subject, it is necessary to have the key which is held by the client.

The method makes it possible to improve the traceability, the privacy and identity-vigilance of analyses. In the case the subject is a human, it also guarantees the client's free will and autonomy as to whether or not to give access to the genomic data is respected, in a stratified manner in relation to different levels of “risk” that may be defined by committees of medical experts.

The DNA label can possess at least one of the following at least three functions:
(1) The labelling (identity-vigilance) of the biological sample by adding a DNA sequence (label) before any pre-analytical treatment. This label can contain a wide variety of data: tube number, date or even any simple and relevant information that allows for the identity-vigilance and traceability of the biological sample throughout the analysis or production chain;
(2) In the case of a patient, the annotation of electronic health record (EHR) patient data via the manufacture of the physical medium in the form of an artificial DNA sequence added to the biological sample which will be sequenced at the same time as the genomic data; and
(3) The security (encryption) through the exogenous DNA sequence (label) which is unique and custom-made. It is the physical carrier of the encryption key(s). It is added to the biological sample at the time of collection and is permanently linked to it.

The sequencing of the sample's DNA results in a text file (e.g. “FASTQ”) that contains the sequences of all or part of the subject's genome as well as the related exogenous DNA sequence (tag). At this stage, it is not possible to distinguish between the different sequences.

“FASTQ” format is a text-based format for storing both a biological sequence (usually nucleotide sequence) and its corresponding quality scores. Both the sequence letter and quality score are each encoded with a single ASCII character for brevity.

Each fragment from the text file (e.g. “FASTQ”) is compared with a reference genome (e.g. human genome databases when the subject is a human). The fragments are aligned with reference sequences (e.g. “hg19”) and fragmented in several “blocks”. Each block is recorded as a level/category of “risk” according to whether the blocks contain data relevant for the analysis or not. Each level is indexed using the DNA tag and cross-referenced to a reference sequence text-based file (e.g. BAM files) that are categorized, compressed and then encrypted with the encryption key(s).

Therefore, in a particular embodiment, blocks comprising the genomic data to be analysed (e.g. the sequence of a gene of interest) are not encrypted while the blocks that do not comprise the sequence of interest are encrypted by means of the encryption key of the DNA tag. In another particular embodiment, blocks comprising the relevant sequences are encrypted by means of a second encryption key (public key), encoded in the DNA tag.

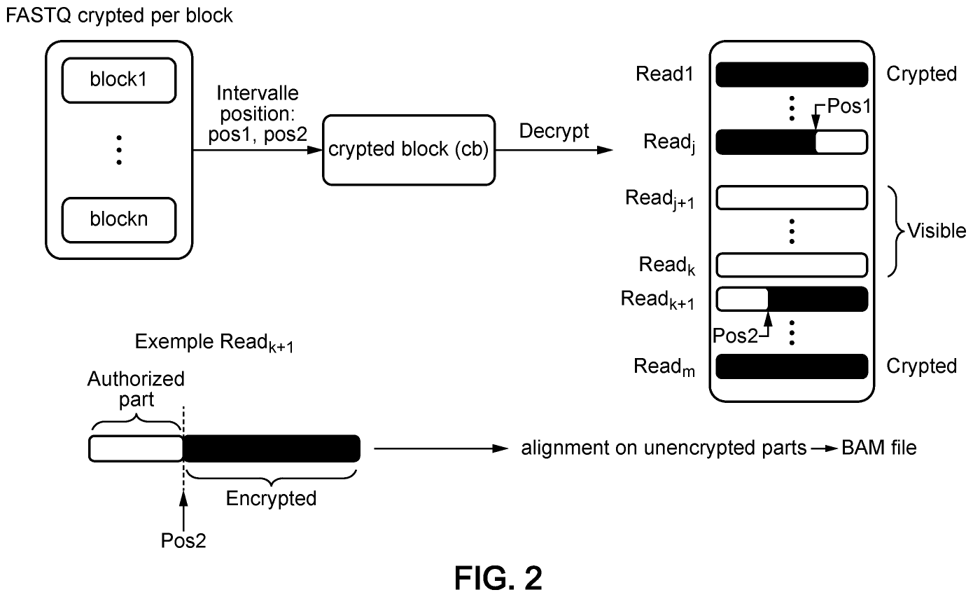

In another particular embodiment, when a block comprises a sequence of interest (or a part of the sequence of interest) and a sequence to be encrypted, it is possible to define positions on the whole sequence of this block so as to encrypt the block, except the sequence of interest. The sequence of interest can furthermore be encrypted by means of the second encryption key so that only this sequence of interest will be deciphered (see FIG. 2).

In a particular embodiment, the encryption of the genome may be subject to the prior agreement of the client, for e.g. by means of a two-factor authentication interface, a smartphone app, a sms, an email, an internet link, etc.

For each subject, information such as at least a database index, the at least one public key and the at least one private key are stored in a file encrypted with a key provided and entered by the client. The client keeps this information in the form of a computer file that is processed by specific software (e.g. KeePass). The index refers to a private database containing information such as for e.g. identity of subject, conditions of sampling, medical records, sequences of interest, etc. Each index is unique and refers specifically to only one subject of this database.

Therefore, the identity of the subject is preserved. No identity can directly be derived from the sampling material. Moreover, only the sequences for which the client agrees to disclose the content are visible for a third-part (e.g. a laboratory in charge of an analysis) while the rest of the genome is protected.

The DNA label is thus the physical and digital medium that allows the genome to be unlocked in a secure manner according to client needs and choice.

A system for implementing the method described above is also provided. Said system comprises a DNA synthesizer configured to synthetize an exogenous DNA sequence corresponding to the DNA tag of the method described above. Therefore, it is possible to encode metadata relating to said subject on the DNA tag. Said metadata comprise at least an encryption key, said encryption key being unique and associated to said subject.

The system further comprises a DNA sequencer configured to sequence said DNA tag. Therefore, at the time of sequencing the DNA of the collected biological sample+the DNA tag, it is possible to sequence the metadata relating to said subject encoded in the DNA tag, and the DNA of said subject.

The system also further comprises least one processing unit configured to create a text-based file corresponding to the sequenced genome of the subject (comprising at least one sequence of interest); then create a text-based file corresponding to the sequenced DNA tag (comprising at least an encryption key); then extract the encryption key from the text-based file of the DNA tag and finally encrypt the text-based file of the genome of the subject with said encryption key.

Preferably, the system further comprise at least one additional processing unit configured to convert the metadata (comprising at least an encryption key) into a binary code based on the combination of the 4 nucleotide bases A, T, G and C so as to obtain a nucleic acid sequence corresponding to said metadata; and transmit the obtained nucleic acid sequence to the DNA sequencer which will produce the corresponding exogenous DNA sequence (comprising encoded metadata comprising at least said encryption key).

More preferably, the system further comprises at least one processing unit configured to fragment the text-based file corresponding to the sequenced genome of the subject in blocks of fixed-length base pairs.

Each of the above-mentioned processing unit can be different processing units or the same.

### EXAMPLES

A particular embodiment of the present method is provided below.

A patient consults a doctor, who prescribes a DNA analysis. The doctor sends a prescription to a company A, with information concerning the sequences to be analysed.

The company A creates a file for the patient and allocate him at least a database index for identification, and at least a set of public/private encryption key. Company A provides the patient with at least his personal private key. Company A then produces a DNA tag comprising metadata (MDD) encoded therein via a DNA synthesizer, said metadata being linked to the patient, and inserts said DNA tag within the sampling material intended to collect a biological sample of the patient.

The DNA tag encode information by using the 4 nucleotide bases, like the binary coding used in computing, e.g. ‘00’=‘A’; ‘01’=‘T’, ‘01’=‘C’, ‘10’=‘G’. Preferably, the DNA tag encodes at least for information that relates to the identity of the patient, to indications of the sequences (e.g. at least one gene) of the genome intended to be analysed (database index) and a cryptographic encryption key (public key). The DNA tag may further include information relating to the sample collection conditions (e.g. date and place); to the nature of the sample (e.g. blood sample taken from a patient with leukaemia) or even to the patient's medical record.

The sampling material containing the DNA tag is then sent to a laboratory B in charge of collecting the biological sample from the patient; and the sample is collected in said sampling material containing the DNA tag. The DNA tag will thus follow the sample from the patient, therefore ensuring its traceability all along the process. The sampling material comprising the biological sample and the DNA tag is then sent back to the company A in order to be sequenced.

The sampling material is sequenced by means of a DNA sequencer in the company A which provides raw text data (e.g. “FASTQ” data) corresponding to the genome of the patient. The “FASTQ” file is then fragmented in several “blocks” of definite length by a processing unit. The processing unit also identifies the index comprised within the DNA tag so as to identify which blocks comprise the at least one sequence to be analysed by a laboratory C. Laboratory C can be the same or a different laboratory than laboratory B. The processing unit then encrypt all the sequences other than the at least one sequence of interest. The encryption is made using the encryption key identified within the DNA tag by the processing unit. FIG. 2 represents the encryption method by blocks. This step can be this step may be subject to the prior agreement of the patient, in real time, for example by means of a two-factor authentication interface, a smartphone app, a sms, an email, an internet link, etc.

The partially encrypted file is then aligned by a processing unit with reference sequences of the human genome (e.g. hg19) to obtain a BAM file output for which only the unencrypted sequences are aligned with the reference genome by a processing unit.

The partially aligned BAM file is then transmitted to the laboratory C, which can have access to the unencrypted sequences in order to analyse the pathogenicity or genomic variation of the sequence of interest. Therefore, the laboratory C has access only to the at least one sequence of interest in order to perform the analysis and the rest of the genome remain encrypted.

In an alternative embodiment, a second set of private key/public key is provided, and said second public key is encoded within the DNA tag. The processing unit then encrypt all the sequences other than the at least one sequence of interest with the first public key and encrypt the sequence of interest with said second public key. Therefore, the file transmitted to a third-part is totally encrypted, providing protection against hacking during the transfer; and said third-part is only able to decipher said sequence of interest but not the rest of the genome.

1. A computer implemented method for encrypting genetic data of a subject, comprising the following steps:
Step a) synthesizing, by a DNA synthesiser, an exogenous DNA sequence comprising encoded metadata relating to said subject, said metadata comprising at least an encryption key, said encryption key being unique and associated to said subject;
Step b) collecting a biological sample of said subject in a sampling material, said sampling material comprising said exogenous DNA sequence;
Step c) sequencing, by a DNA sequencer, the DNA of said subject obtained from said biological sample and sequencing, by a DNA sequencer, said exogenous DNA sequence comprising encoded metadata,
Step d) creating by at least one processing unit a text-based file corresponding to the sequenced genome of the subject, said genome comprising at least one sequence of interest,
Step e) creating by said least one processing unit a text-based file corresponding to the sequenced exogenous DNA sequence comprising encoded metadata comprising at least an encryption key;
Step f) extracting by means of said least one processing unit the encryption key from said text-based file corresponding to the sequenced exogenous DNA sequence;
Step g) encrypting by said least one processing unit said text-based file corresponding to the sequenced genome of the subject with said encryption key from step f) associated to said subject, apart from the at least one sequence of interest.

2. The method according to claim 1 wherein in step a, said metadata comprise at least a second encryption key and in step g, the at least one sequence of interest is encrypted by means of said second encryption key.

3. The method according to claim 1 wherein the text-based file of step d) is fragmented in blocks of fixed-length base pairs.

4. The method according to claim 1, including encoding a personal database index identifier associated to said subject within the exogenous DNA sequence.

5. The method according to claim 1, including encoding information to identify the at least one sequence of interest within the exogenous DNA sequence.

6. The method according to claim 1, wherein the subject is a patient and including encoding the health record of the subject within the exogenous DNA sequence.

7. The method according to claim 1, including encoding metadata in the exogenous DNA sequence in the form of a binary code based on the combination of the 4 nucleotide bases A, T, G and C.

8. The method according to claim 1, including encrypting the metadata encoded within the exogenous DNA sequence with a third encryption key.

9. A system for encrypting genetic data of a subject, comprising:
(a) a DNA synthesizer configured to synthetize an exogenous DNA sequence comprising encoded metadata relating to said subject, said metadata comprising at least an encryption key, said encryption key being unique and associated to said subject;
(b) a DNA sequencer configured to sequence said exogenous DNA sequence comprising encoded metadata relating to said subject and configured to sequence the DNA of said subject obtained from a biological sample;
(c) at least one processing unit configured to perform the following steps:
creating a text-based file corresponding to the sequenced genome of the subject, said genome comprising at least one sequence of interest;
creating a text-based file corresponding to the sequenced exogenous DNA sequence, the sequence of said exogenous DNA sequence comprising encoded metadata comprising at least an encryption key;
extracting the encryption key from the text-based file corresponding to the sequenced exogenous DNA sequence;
encrypting the text-based file corresponding to the sequenced genome of the subject with said encryption key.

10. The system according to claim 9, comprising at least one additional processing unit configured to perform the following steps:
convert the metadata comprising at least an encryption key into a binary code based on the combination of the 4 nucleotide bases A, T, G and C so as to obtain a nucleic acid sequence corresponding to said metadata;
transmitting the obtained nucleic acid sequence to the DNA sequencer so as to obtain the exogenous DNA sequence comprising encoded metadata comprising at least said encryption key.

11. The system according to claim 9, wherein said at least one processing unit is further configured to fragment the text-based file corresponding to the sequenced genome of the subject in blocks of fixed-length base pairs.
